(19) United States
(12) Patent Application Publication
(10) Pub. No.: US 2022/0144904 A1
(43) Pub. Date: May 12, 2022

(54) Composition for and Method of Improving Tissue Performance

(71) Applicant: OHIO STATE INNOVATION FOUNDATION, Columbus, OH (US)

(72) Inventors: Jianjie MA, Powell, OH (US); Tao TAN, Columbus, OH (US); HUA ZHU, Columbus, OH (US)

(73) Assignee: OHIO STATE INNOVATION FOUNDATION, COLUMBUS, OH (US)

(21) Appl. No.: 17/583,549

(22) Filed: Jan. 25, 2022

Related U.S. Application Data
(63) Continuation of application No. PCT/US2020/038104, filed on Jun. 17, 2020.
(60) Provisional application No. 62/878,538, filed on Jul. 25, 2019.

(51) Int. Cl. C07K 14/47

(52) U.S. Cl. CPC C07K 14/4702; A61K 38/00

(57) ABSTRACT

Compositions for and methods of improving tissue function are provided. Said compositions comprise MG53 or express MG53. Said compositions can be used for improving the function of non-diseased and uninjured tissue in subjects.

### CROSS-REFERENCE TO EARLIER FILED APPLICATIONS

This application is a continuation of application No. PCT/US2020/038104 filed Jun. 17, 2020, which claims the benefit of U.S. application No. 62/878,538 filed Jul. 25, 2019, the entire disclosures of which are hereby incorporated by reference.

### STATEMENT REGARDING FEDERALLY SPONSORED RESEARCH

The invention was made with government support under R01 AR061385 awarded by the National Institutes of Health, R01 AG056919 awarded by the National Institutes of Health, R01 DK106394 awarded by the National Institutes of Health, R44 DK112403 awarded by the National Institutes of Health, and R44 GM123887 awarded by the National Institutes of Health. The government has certain rights in this invention

### FIELD OF THE INVENTION

The present invention concerns compositions for and methods of improving the performance of tissue. In particular, it concerns improving the performance of muscle tissue and organ tissue that is otherwise healthy, meaning neither diseased nor acutely injured.

### BACKGROUND OF THE INVENTION

MG53 protein (also referred to as mitsugumin 53 or TRIM72) is known in the art: U.S. Pat. No. 7,981,866, WO2008/054561, WO2009/073808, US2011/0202033, US2011/0287004, US2011/0287015, US2013/0123340, WO2011/142744, WO2012/061793, U.S. Pat. Nos. 8,420,338, 9,139,630, 9,458,465, 9,494,602, US2014/0024594, WO2012/134478, WO2012/135868, US2015/0110778, WO2013/036610, US2012/0213737, WO2016/109638, the entire disclosures of which are hereby incorporated by reference. Therapeutic uses thereof are described in the art.

MG53 is present in serum derived from the blood of mice, rats, and humans (Zhu H, et al., “Amelioration of ischemia-reperfusion-induced muscle injury by the recombinant human MG53 protein” in Muscle & nerve (2015), 52, 852-858; and Liu J, et al., “Cardioprotection of recombinant human MG53 protein in a porcine model of ischemia and reperfusion injury” in Journal of molecular and cellular cardiology (2015), 80, 10-19, the entire disclosures of which are hereby incorporated by reference). MG53 is predominantly expressed in skeletal and cardiac muscle; however, the amount of MG53 present or the level of MG53 expression that occurs in tissue is often insufficient to overcome reduced performance of otherwise healthy tissue.

MG53 has been reported to be useful for repairing some acutely injured tissue (such as by physical injury) or some chronically injured tissue (such as by disease). U.S. Pat. Nos. 7,981,866 and 9,139,630 suggest that MG53 can be used to treat many conditions, diseases and disorders; however, the present inventors have determined that said art is unduly broad and have experimentally identified diseases, conditions and injury types for which exogenous administration of MG53 has been found to be therapeutically ineffective, e.g. multiple sclerosis, viral infection, radiation induced tissue injury, and obesity.

MG53 has not been reported to improve the performance of otherwise healthy tissue exhibiting reduced performance; however, reduced performance of tissue is known to occur even though the etiology of such reduction might not be understood. It would be an advancement in the art to improve tissue performance of non-diseased non-acutely injured tissue.

### SUMMARY OF THE INVENTION

The present invention seeks to provide compositions for and methods of improving the performance of tissue, in particular, improving the performance of otherwise healthy tissue that is neither diseased nor acutely injured. The present invention provides unexpected improvements supported by based upon MG53-related data undisclosed in the prior art.

Our findings show that administration of exogenous MG53 improves tissue performance, in particular of skeletal muscle tissue, of organ tissue, such as cardiac muscle tissue, of non-muscle tissue, such as kidney, and of neuronal tissue. This is particularly unexpected when said tissue is healthy or otherwise not diseased.

In some embodiments, administration of exogenous MG53 improves contraction of muscle tissue, improves Ca²⁺ signaling in muscle tissue, improves muscle satellite cell proliferation, improves recovery of strenuously exercised muscle, and/or improves overall performance of muscle tissue.

In some embodiments, administration of exogenous MG53 improves cardiac output function, e.g. improves left ventricular ejection volume.

In some embodiments, administration of exogenous MG53 provides a reduction in kidney resident immune cell activation, reduction in serum level of creatinine, reduction in collagen deposition, reduction in macrophage infiltration, and/or increased kidney tubular health.

In some embodiments, administration of exogenous MG53 improves neuromuscular junction function, and/or improves brain function.

In some embodiments, exogenous administration of MG53 increases the lifespan of a subject as compared to the projected average lifespan of other healthy subjects of the same species.

An aspect of the invention provides a method of improving tissue function, the method comprising administering to a subject an effective amount of exogenous MG53. In some embodiments, the tissue is not suffering from a disease. In some embodiments, the tissue has not been physically injured, such as by impact force or cutting. In some embodiments, the tissue is otherwise healthy except for exhibiting reduced (impaired) function as compared to other similar tissue. In some embodiments, the tissue is healthy and administration of MG53 improves function (performance) of said tissue as compared to function (performance) prior to administration of said tissue.

Another aspect of the invention provides a method of improving tissue function in a subject exhibiting increased intracellular aggregation of MG53 in tissue of said subject, the method comprising administering to a subject with impaired MG53 function, i.e. a subject exhibiting increased level of intracellular aggregation of MG53 in said tissue, a composition comprising exogenous MG53 (and optionally at least one antioxidant and/or at least one other active ingredient). As used herein, said subject would typically exhibit intracellular aggregation of MG53 of at least 10% of the total amount or concentration of intracellular MG53 in said tissue.

Another aspect of the invention provides a method of improving tissue function in subjects exhibiting elevated levels of intracellular MG53 aggregation in said tissue, the method comprising chronically administering to said subject MG53 over a treatment period of at least 2-6 weeks. In some embodiments, the chronic administration is at least one weekly, at least once daily, two or more times daily, two or more times per week, at a dosing range from 0.01 mg/kg to 20 mg/kg rhMG53 protein per body weight.

Another aspect of the invention provides a method of improving athletic performance in a human or animal subject, the method comprising at least the following step(s): prior to conducting an athletic activity, administering to said subject an effective amount of MG53, whereby said administering results in improved athletic performance of said subject as compared to said subject's performance in said athletic activity when not administered MG53. The method can further comprise the step of said subject conducting said athletic activity. Said administration can be acute or chronic. The MG53 can be administered as described herein. One or more other active ingredients can also be administered to said subject to further improve athletic performance.

Another aspect of the invention provides a method of improving recovery of strenuously exercised muscles in a human or animal subject, the method comprising at least the following step(s): prior to or after conducting a strenuous athletic activity, administering to said subject an effective amount of MG53, whereby said administering results in improved recovery of said strenuously exercised muscle as compared to recovery when said subject is not administered MG53. The method can further comprise the step of said subject conducting strenuous exercise. Said administration can be acute or chronic. The MG53 can be administered as described herein. One or more other active ingredients can also be administered to said subject to further improve said recovery of strenuously exercised muscle.

Exemplary animals that can be treated with MG53 (alone or along with one or more other active compounds) include horses, dogs, or cats.

In some embodiments, the method of the invention further comprises adjunct therapy or co-therapy with at least one antioxidant, whereby said at least one antioxidant is administered prior to, along with, or after administration of MG53. Accordingly, the method of the invention can further comprise the step of administering at least one antioxidant to a subject. In some embodiments, the invention provides a method of improving tissue function in a subject, the method comprising chronically administering to said subject MG53 and at least one antioxidant. The molar ratio of MG53 to antioxidant can be in the range of 0.01 to 10.

MG53 is administered chronically to improve tissue function. In some embodiments, exogenous MG53 is administered systemically. Systemic administration of MG53, in particular recombinant human MG53 (rhMG53), improves tissue function. rhMG53 administered to the circulatory system (e.g. blood) can translocate to other tissue, whereby it enters said tissue and improves its performance (function) as compared to its performance prior to said administration.

When administered prophylactically, MG53 prevents the reduction of tissue performance or reduces the rate of reduction of tissue performance over time as compared to other subjects of the same demographic description as the subject to which MG53 is administered.

A composition of the invention can be administered one, two, three or more times per day. It can be administered daily, weekly, monthly, bimonthly, quarterly, semiannually, annually or even longer as needed. It can be administered every other day, five times per week, four times per week, three times per week, two times per week, once daily, twice daily, one to four times daily, continuously, or as frequently or infrequently as needed. The unit dose of each administration is independently selected upon each occurrence from the doses described in this specification or as determined to be therapeutically effective. All combinations of the dosing regimens described are contemplated to be within the scope of the invention. A dose of about 0.01 to about 10 mg of MG53 per kg of body can be used and administered according to the frequencies described herein.

The MG53 can be administered systemically, e.g. intramuscularly, intravenously, intraperitoneally, subcutaneously, orally, via inhalation, enterically, or a combination of two or more thereof. The MG53 can also be administered topically or topically in combination with systemically.

Another aspect of the invention provides a dosage form that releases or provides MG53. The dosage form can be a non-biological dosage form or a biological dosage form. Suitable dosage forms release or provide MG53 to the target tissue either directly or indirectly.

A dosage form can be a spray, powder, cream, ointment, liquid, gel, solution, suspension, implant, explant, tablet, pill, sachet, bead(s), pellet(s), osmotic device or other pharmaceutically acceptable dosage form.

Another aspect of the invention provides a biological dosage form that releases MG53 or enables expression of MG53 followed by release of MG53. A biological dosage form is one whose primary carrier or medium or content is a biological product. Suitable biological dosage forms include: a) viral vector, adenoviral vector, or retroviral vector that enters the target tissue or circulatory system and causes expression and release of MG53, whereby said target tissue is treated with MG53; b) autologous blood serum comprising added exogenous MG53; c) autologous blood serum comprising viral vector, adenoviral vector, or retroviral vector that causes expression of MG53 in cellular tissue; d) autologous blood serum comprising bioengineered hematopoietic stem cells that express and release MG53; or e) a combination of any two or more of the above.

The invention also provides an autologous serum dosage form comprising exogenously added MG53. The invention also provides an autologous serum dosage form comprising cells that express MG53. The invention also provides an autologous serum dosage form comprising a viral vector that causes cells to express MG53.

Another aspect of the invention provides a co-therapeutic or adjunctive method of improving tissue performance, the method comprising administering to a subject in need thereof (meaning a subject with tissue exhibiting reduced performance even though said tissue is non-diseased and uninjured) an effective amount of MG53 and an effective amount of one or more other active ingredients, which are suitable for tissue performance. Exemplary other active ingredients include growth hormone(s), anti-inflammatory agent(s), anti-fibrotic agent(s), immunomodulator agent(s), compound(s) that improves integrity of muscle fiber, compound(s) that improves MSC, or a combination thereof. MG53 and said one or more other active ingredients can be administered simultaneous, sequentially or in an overlapping manner.

The dosage form is independently selected at each occurrence. A combination of two or more different dosage forms can be administered to the subject in need. Two or more different modes of administration can be employed.

In some embodiments, the dosage form further comprises one or more zinc salts present in an amount sufficient to promote or enhance said improvement of tissue performance by exogenously administered MG53. In a composition of the invention, the molar ratio of Zn ions present to MG53 molecules present is at least 2 to 1, when considering the two zinc ion binding sites present on each MG53 molecule. In some embodiments, the composition comprises a molar ratio of >2:1 for the moles of Zn to moles of MG53.

In some embodiments, a subject is chronically administered MG53, at least one antioxidant, and at least one zinc salt. The invention also provides a composition comprising MG53, at least one antioxidant, and at least one zinc salt. The invention also provides a method of improving tissue function, the method comprising chronically administering a composition comprising MG53, at least one antioxidant, and at least one zinc salt.

Embodiments of the invention exclude compositions comprising single unaltered natural product; however, said compositions may comprise mixtures of said unaltered natural product(s) along with other components thereby resulting in manmade compositions not present in nature. Embodiments of the invention exclude processes that employ solely unaltered natural processes; however, said processes may comprise a combination of said unaltered natural processes along with one or more other non-natural steps, thereby resulting in processes not present in nature. Embodiments of the invention may also include new uses (new methods of treatment) for natural products, new compositions comprising said natural products, and new methods employing said natural products.

The invention includes all combinations of the aspects, embodiments and sub-embodiments disclosed herein. Other features, advantages and embodiments of the invention will become apparent to those skilled in the art by the following description, accompanying examples and appended claims.

### BRIEF DESCRIPTION OF THE FIGURES

The following drawings are part of the present specification and are included to further demonstrate certain aspects of the invention. The invention may be better understood by reference to one or more of these drawings in combination with the detailed description of the specific embodiments presented herein.

FIG. 1 depicts photomicrographs of immunohistochemically stained mouse and human skeletal muscle exhibiting normal and increased levels of intracellular aggregation of MG53.

FIG. 2 depicts photomicrographs of Western Blot gels for quantifying the serum level of MG53 before and after mild running exercise (10 m/min for 1 hour) in normal mice as compared to mice exhibiting increased levels of intracellular aggregation of MG53.

FIG. 3 depicts single fiber electromyographs to measure muscle jitter in normal mice and impaired mice exhibiting impaired neuromuscular junction (NMJ) function.

FIG. 4 depicts a chart of muscle jitter in the impaired mice (of FIG. 3) after treatment with control vehicle or with MG53 in vehicle.

FIG. 5 depicts a chart of the contractile muscle strength in the impaired C57BL/6J mice (FIG. 3) after treatment with MG53 (6 mg/kg, subcutaneous, daily) over a six-week treatment period.

FIG. 6 depicts photomicrographs of stained neuromuscular junction in Group 2 mice with and without treatment with rhMG53.

FIG. 7 depicts photomicrographs establishing improvement of MG53 aggregates in skeletal muscle derived from Group 2 mice following treatment with antioxidant, N-acetyl cysteine.

FIG. 8 depicts a chart of dose-dependent response in the fraction of maximal muscle force recovered in the plantarflexors of C57BL/6NJ mice when rhMG53 was administered in the indicated dose at four hours after completion of repeated stimulation.

FIG. 9 depicts a chart of time-dependent response in the fraction of maximal undamaged limb force (meaning contractility) recovered in mice receiving a first dose applied at 24 hours after stimulation (day—1; 6 mg/kg, subcutaneous: S.C.) and then daily doses (6 mg/kg, subcutaneous daily) of rhMG53 over a 28-day dosing period as compared to administration of control vehicle.

FIG. 10A depicts photomicrographs of Western Blot gels for quantifying the level of MG53 expression in heart muscle derived from two mice groups (Group 1: 3 month; Group 2: 20 month).

FIG. 10B depicts a chart of the left ventricular (LV) ejection fraction (EF) of young and elderly mice of FIG. 10A.

FIG. 10C depicts a chart of quantification of the level of expression of endogenous MG53 in the mice of FIG. 10A, wherein the mice of Group 2 exhibit reduced level of MG53 expression.

FIG. 11 depicts charts of time dependent changes in heart rate, left ventricular (LV) ejection fraction (EF), fraction shortening, and cardiac output of the mice following treatment with repetitive treatment with rhMG53 (6 mg/kg, daily subcutaneous) for 6 weeks in Group 2 mice.

FIG. 12 depicts a chart of the changes in serum level of creatinine in Group 2 mice following treatment of rhMG53, demonstrating the benefits of rhMG53 to improve kidney function in aging.

FIG. 13 depicts a chart of the number of dropouts versus running speed for wild-type mice and tPA-MG53 mice.

FIG. 14 depicts a chart of the total number of meters run over the indicated number of days for wild-type mice and tPA-MG53 mice.

FIG. 15 depicts measurement of intracellular Ca using a fluorescent indicator in muscle fibers obtained from wild type and tPA-MG53 mice.

FIG. 16 depicts a chart comparing the half-time of Ca decay in wild type and tPA-MG53 mice.

FIG. 17 depicts measurement of intracellular Ca in THP-1 cells testing the effect of rhMG53 protein.

FIG. 18 depicts photomicrographs of single extensor digitorum longus (EDL) muscle fibers from wild type (WT) mice, tPA-MG53 mice, MG53 knockout (KO) mice, and KO mouse muscle cultured in the presence of rhMG53, for characterization of the growth of muscle satellite cells.

FIG. 19 depicts the quantification of muscle satellite cell growth in muscle derived from FIG. 18.

### DETAILED DESCRIPTION OF THE INVENTION

Unless specified otherwise, all embodiments of the invention comprising or employing “MG53” include all known forms of MG53. It also refers to recombinant human MG53 (rhMG53, for example as described in Example 1).

As used herein and unless otherwise specified, the term MG53 protein refers to the MG53 protein present as the native form, optimized form thereof, mutant thereof, derivative thereof or a combination of any two or more of said forms. Native MG53 contains 477 amino acids that are well conserved in different animal species. Methods of preparing and/or isolating MG53 are known: U.S. Pat. No. 7,981,866, WO2008/054561, WO2009/073808, US2011/0202033, US2011/0287004, US2011/0287015, US2013/0123340, WO2011/142744, WO2012/061793, U.S. Pat. Nos. 8,420,338, 9,139,630, 9,458,465, 9,494,602, US2014/0024594, WO2012/134478, WO2012/135868, US2015/0110778, WO2013/036610, US2012/0213737, WO2016/109638, the entire disclosures of which, including sequence information therein, are hereby incorporated by reference.

The sequence listing information for native MG53, and variants or various forms thereof, is disclosed in U.S. Pat. Nos. 7,981,866 and 9,139,630, the entire disclosures of which, including sequence information therein, are hereby incorporated by reference. The sequence listing information for a cDNA that encodes optimized native human MG53, or a fragment thereof, is disclosed in U.S. Pat. No. 9,139,630, the entire disclosure of which, including sequence information therein, is hereby incorporated by reference.

As used herein in reference to MG53, the term “mutant” means a recombinant form of MG53 having an amino acid change (replacement) of one, two, three or more amino acids in the amino acid sequence of native MG53. Mutant forms of MG53 and methods of preparing the same are known: US2015/0361146, EP3118317, WO2015/131728, U.S. Pat. No. 9,139,630, the entire disclosures of which, including sequence information therein, are hereby incorporated by reference.

As used herein the term “endogenous MG53”, refers to MG53 present in a subject prior to treatment with a composition, dosage form, or method according to the invention. As used herein, exogenous MG53 is nonendogenous MG53.

The present inventors have unexpectedly discovered that healthy tissue sometimes exhibits elevated levels of intracellularly aggregated MG53 and results in reduced or impaired cell function. Said elevation can be non-disease-related. The present inventors have determined that impaired function even in non-diseased tissue can be improved regardless of whether or not said tissue already expresses MG53 naturally. We also determined that the amount of endogenous MG53 provided by the body is on its own insufficient to overcome impairment of tissue function. It is unexpected that administration of exogenous rhMG53 to said tissue would provide a clinical benefit to and/or improve the performance of said tissue. It is also unexpected that administration of MG53 and at least one antioxidant would provide a clinical benefit to and/or improve the performance of said tissue.

We also determined that long-term (chronic, repeated) administration of exogenous MG53 (and optionally at least one antioxidant, and optionally at least one zinc salt) to a subject unexpectedly increases the lifespan of said subjects as compared to the average lifespan of a population of demographically similar subjects not administered the MG53. In some embodiments, said subjects are healthy. In some embodiments, the subjects are diseased.

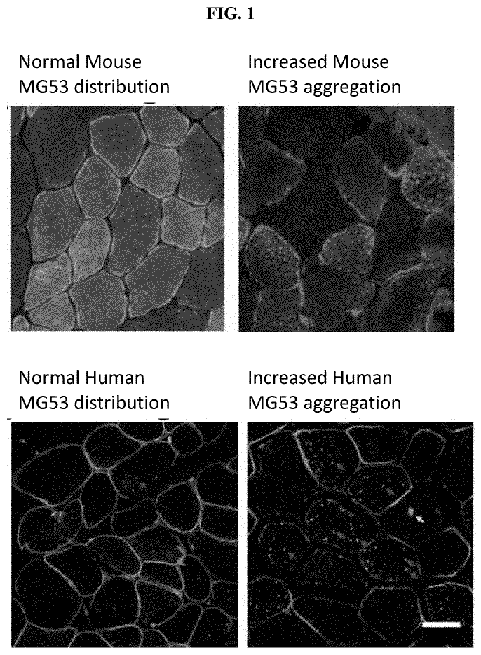

Pilot studies were conducted to explore the impact of exogenously administered MG53 upon muscle cells exhibiting impaired-MG53 function. Skeletal muscle tissue from healthy wild type mice was divided into two groups (Group 1: 3-6 month; Group 2: 24-27 month) and immunohistochemically stained to quantify the respective levels of intracellular aggregated MG53. It was discovered that Group 2 mice exhibit impaired MG53 function because they exhibited increased levels of intracellularly aggregated MG53 as seen via photomicrographic analysis (FIG. 1, Example 2).

Similar level of MG53 aggregation was observed in skeletal muscle derived from human biopsy samples in an elderly population of subjects.

The performance of impaired and un-impaired skeletal muscle was compared by performing exercise tests on the above healthy subjects. The mice underwent prolonged exercise tests by being subjected to voluntary wheel running for a period of 30 days. The Group 2 mice ran for 1.6 miles/day and the Group 1 mice ran for 8 miles/day.

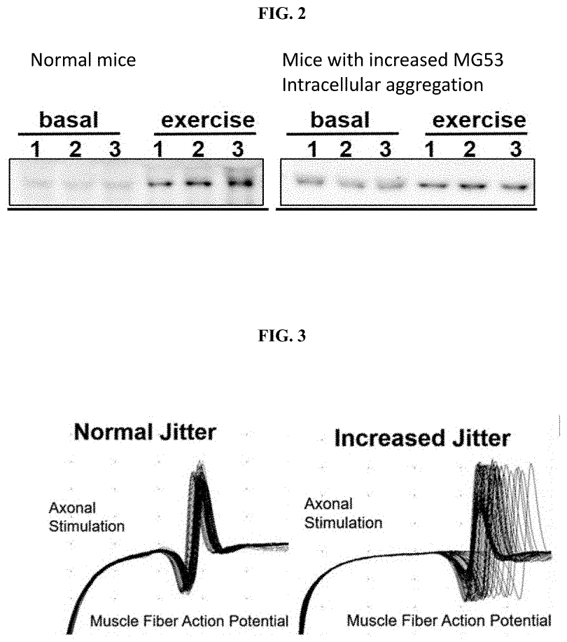

When the serum levels of MG53 in the mice in the resting state was determined and quantified by Western Blot analysis (FIG. 2, Examples 3), it was found that the Group 2 mice surprisingly had higher serum levels of MG53; however, when the mice were subjected to mild exercise in a wheel (10 m/min for 1 hour), we unexpectedly found that the serum level of MG53 of the Group 1 mice increased, whereas it remain essentially unchanged for the Group 2 mice. We, thus, expected that administration of exogenous MG53 would not provide any clinical benefit to the Group 2 mice because of their already elevated serum levels of MG53.

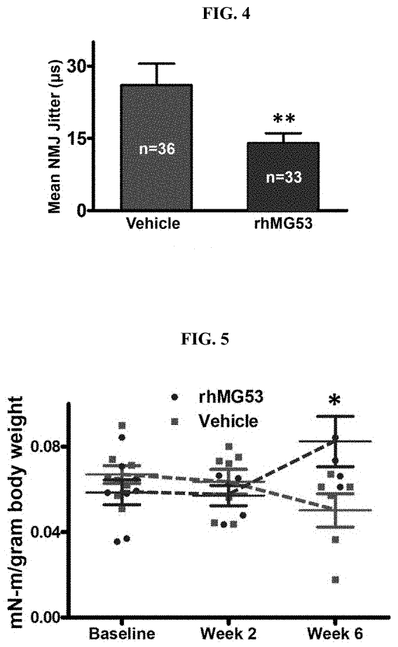

We found, however, that administration of exogenous MG53 to the Group 2 mice improves neuromuscular junction function. The mice above were subjected to single fiber electromyography (SFEMG) tests (Example 4) to quantify NMJ function. As depicted in FIG. 3, the Group 1 mice exhibited normal low levels of jitter; whereas, the Group 2 mice exhibited high levels of jitter even though they have higher levels of MG53 in serum than do the Group 1 mice. We then treated the Group 2 mice with rhMG53 (daily administrations 6 mg/kg subcutaneous) or with control vehicle over a 6-week period and quantified mean NMJ jitter. Contrary to our expectations, we found (FIG. 4, Example 5) that the Group 2 mice exhibited improved muscle function as proven by a substantial reduction in jitter.

We determined that improved muscle function was also obtained in terms of reducing the rate of decline of contractile force (Example 6). The results in FIG. 5 indicate that muscle contractile force was increased in the impaired mice during the 6-week treatment period with systemically administered exogenous rhMG53. Again, this was unexpected because the impaired mice already have increased serum and intracellular levels of MG53 as compared to unimpaired mice.

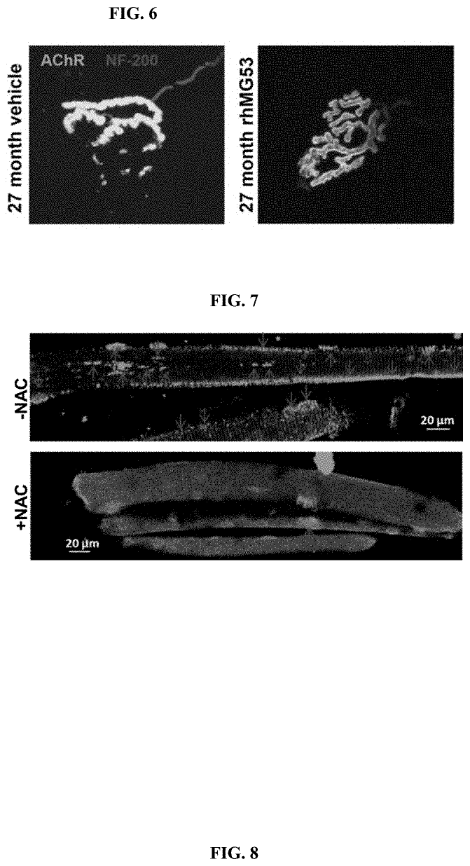

We performed immunohistochemical staining with skeletal muscle derived from the Group 2 mice (Example 7), with or without treatment with rhMG53, to characterize the changes in the integrity of neuromuscular junction (NMJ). The picture in FIG. 6 indicate that rhMG53 treatment led to improved integrity of NMJ in the Group 2 muscle.

We treated mice that have aggregates of intracellular MG53 with N-acetyl cysteine, an antioxidant, and found that this treatment lead to improved MG53 function as evidenced by the more homogenous distribution of MG53 in skeletal muscle. This is depicted in FIG. 7.

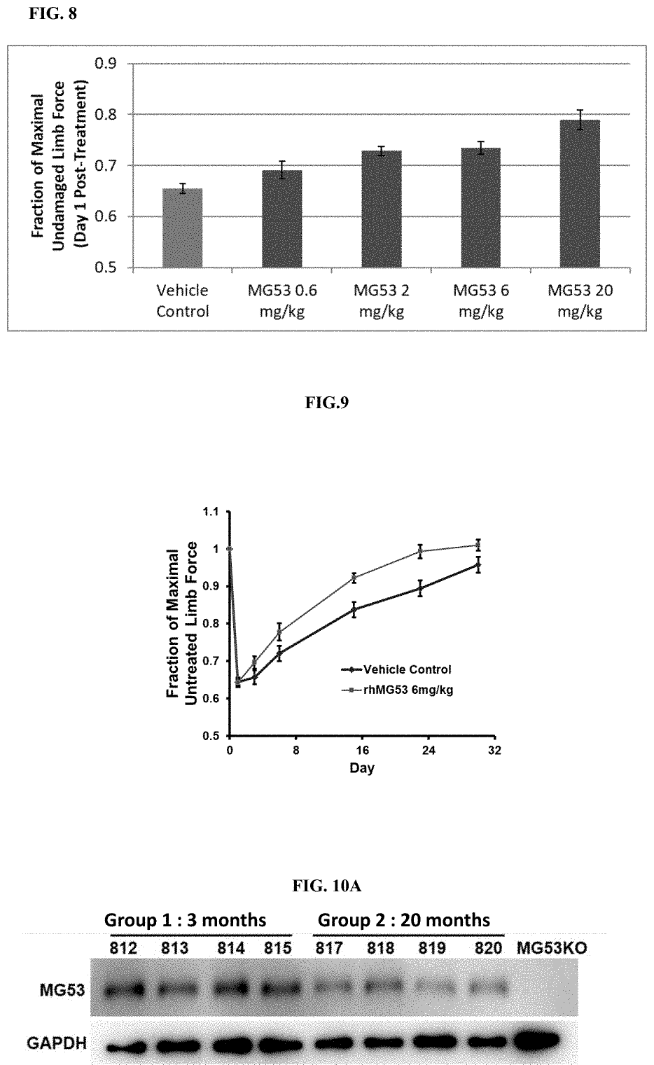

Further evidence of the efficacy of administration of exogenous MG53 toward improvement of performance of muscle tissue was obtained by conducting hind-limb plantar flexor muscle contraction studies on 12-week old C57BL/6N mice (Example 8), which were stimulated to produce tetanic contractions (via the sciatic nerve; 60 repetitions; 10 second apart each) while being forcibly lengthened. Different doses of rhMG53 were administered at 4 hours after tetanic contractions. The recovered fraction of maximal force of contraction was determined at the indicated doses. The data in FIG. 8 indicates effective (statistically significant) dose-dependent improvement of muscle performance in this assay.

Exogenous rhMG53 administered (intravenous) at doses as low as 0.6 mg MG53/kg body weight to 20 mg/kg were found to be effective in an increasing dose dependent manner. Higher doses can be administered because MG53 is non-toxic.

Even though MG53 is already present in skeletal muscle, we determined that subcutaneous (s.c.) administration of MG53 unexpectedly improves muscle function even at 24 hour following repeated contraction test. Mice, having undergone the contraction test, were treated with a first dose of rhMG53 (2 mg/kg, i.v.) at 24 hours after completion of the repeated contractions and daily (6 mg/kg, s.c.) thereafter for a period of 28 days. FIG. 9 depicts the results obtained for recovered fraction of maximal limb force in a time dependent manner throughout the 28-day period. Even after 28 days, the control vehicle has not recovered 100% of the limb force; whereas, the MG53 treated mice have recovered 98-99% of the limb force at about 20 days. Moreover, it took the vehicle-treated mice 20 days to recover about 90% of the limb force, but it only took about 12 days for the MG53-treated mice to recover about 90% of the limb force. This significant improvement in muscle performance by administration of exogenous MG53 was unexpected, because the mice already have substantial serum and intracellular levels of MG53.

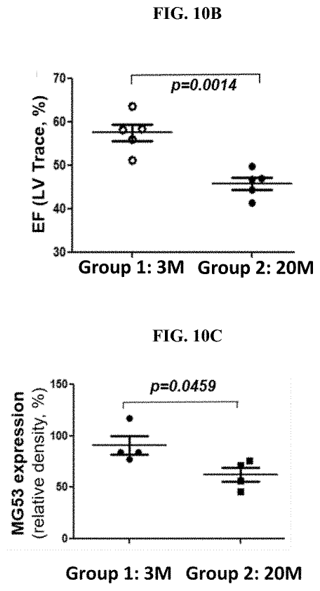

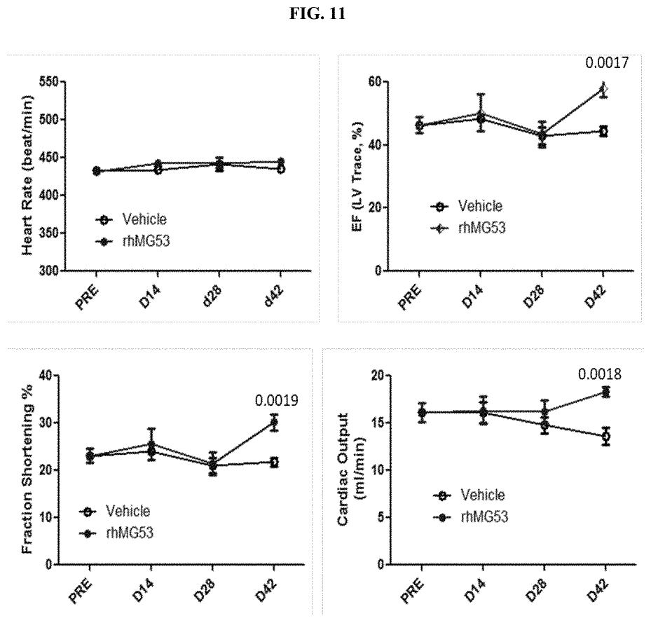

We performed Western blot analysis of endogenous MG53 expression in heart tissue derived from Group 1 and Group 2 mice, and found significant reduction of MG53 in the heart tissue derived from Group 2 mice. Echocardiography measurement revealed significant reduction in ejection fraction (EF) in Group 2 mice. These findings were depicted in FIG. 10.

We then determined that administration of exogenous MG53 can improve the function of heart from the Group 2 mice. Following a 6-week repetitive treatment with rhMG53 (6 mg/kg, s.c. daily), the longitudinal studies demonstrated significant benefits of rhMG53 in preventing the decline of EF in the Group 2 mice supported the notion that Group 2 receiving rhMG53 exhibited healthier heart function compared with those receiving the vehicle saline control. These findings are shown in FIG. 11, Example 9.

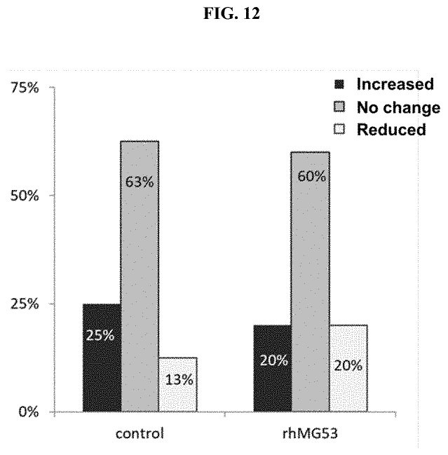

We determined that administration of exogenous rhMG53 to a subject also improves kidney function. Assessment of serum level of creatinine in Group 2 mice subjected to rhMG53 treatment demonstrated improved kidney function in elderly mice, as the proportion of mice with reduced serum creatinine levels was greater than those mice receiving saline as control (FIG. 12, Example 10).

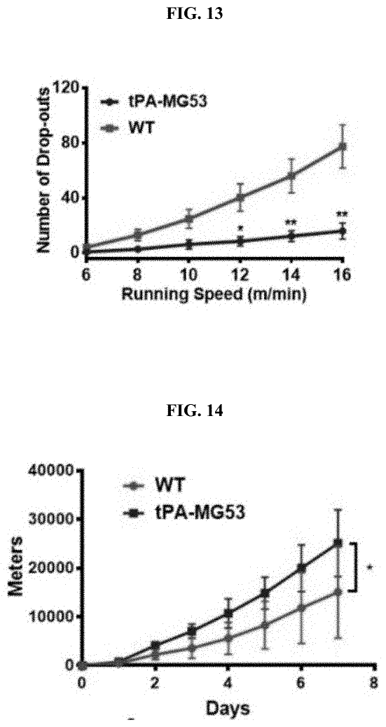

The impact of MG53 upon skeletal muscle performance was then evaluated by comparing wild type (WT) mice to tPA-MG53 mice, which produce increased serum levels of MG53 as compared to WT mice and which serve as a genetic model for systemic administration of exogenous MG53. The mice were subject to a running exercise test (Example 11) and the number of dropouts (FIG. 13) and distance run (meters, FIG. 14) were measured. The tPA-MG53 mice exhibited a substantial reduction in dropouts (indicating improved muscle endurance) and a substantial increase in distance run (indicating improved muscle function).

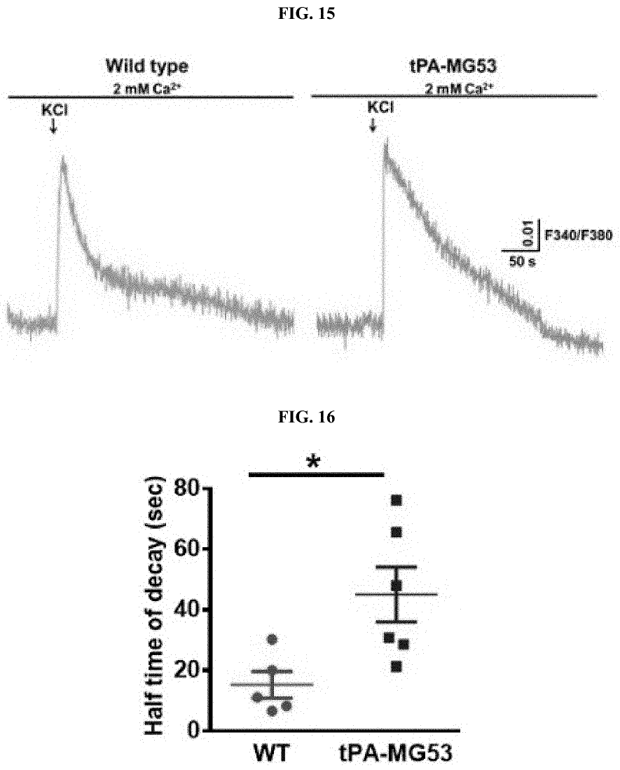

The same groups of mice were subject to determine Ca²⁺ signaling efficiency testing (Example 12). It was observed that skeletal muscle derived from the tPA-MG53 mice exhibited substantially improved Ca²⁺ signaling (FIG. 15) with substantially increased half-time of decay (FIG. 16), thus establishing that systemic administration of MG53, at least at the doses tested, results in improved performance of muscle tissue.

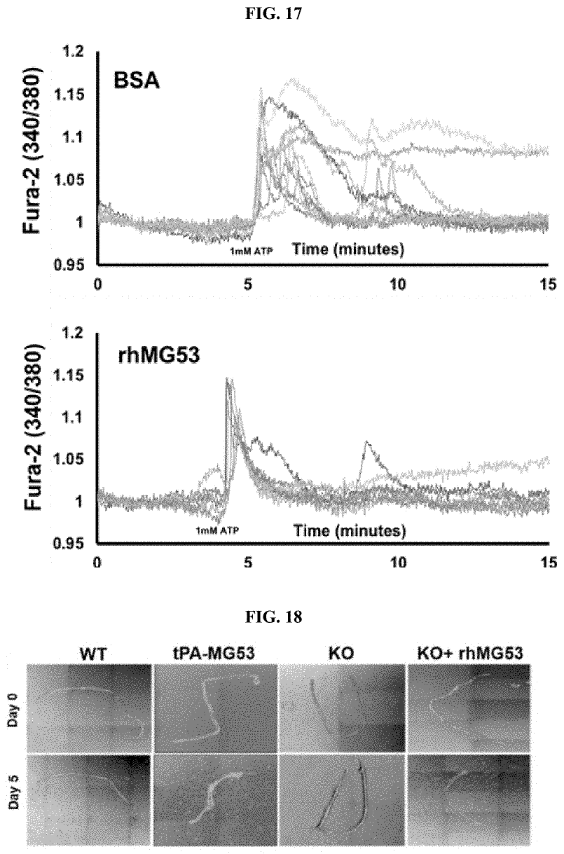

The improvement in multi-tissue function may also reflect the effect of MG53 in controlling inflammation and macrophage function. We used THP-1 cells as a model of human macrophage study. As depicted in FIG. 17, treatment of THP-1 cells with ATP led to release of intracellular Ca from the endoplasmic reticulum, as evidenced by the transient increase of fluorescent-Ca indicator signal. Compared with cells treated with bovine serum albumin (BSA) as control, addition of rhMG53 (1 ug/ml) lead to significant suppression of intracellular Ca signaling. This unexpected finding provides direct support for a role for MG53 in control of macrophage function.

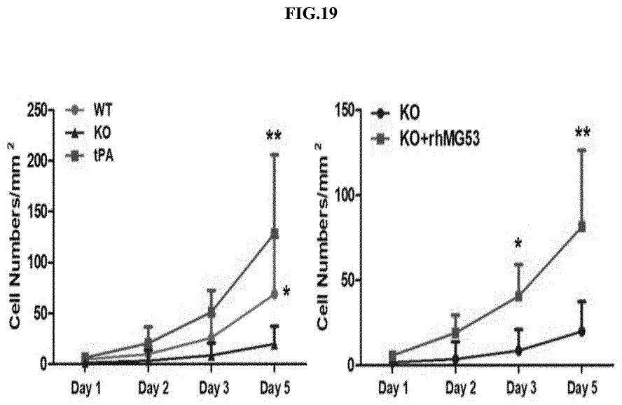

Further evidence of the improvement of performance in healthy muscle tissue was obtained by determining muscle satellite cell (MSC) proliferation in the presence and absence of rhMG53 (FIG. 18, Example 13). Isolated EDL muscles (myofiber) were obtained from WT mice, tPA-MG53 mice, and KO mice and cultured. At 5 days after initiation of culture, the muscle tissue from KO mice exhibited substantially fewer MSC's than the tissue from WT mice and tPA-MG53 mice. The KO myofiber was then incubated with rhMG53 (20 μg/ml) which led to an increase in the number of MSC's near the myofiber indicating that administration of MG53 improves muscle tissues ability to proliferate MSC's. Statistical analysis demonstrated the beneficial effects of rhMG53 in promoting MSC proliferation (FIG. 19). This unexpected finding provides a base for the role of MG53 in the long-term improvement of skeletal muscle function.

Accordingly, the invention provides a method of improving muscle satellite cell proliferation in muscle tissue of a subject, the method comprising administering to said subject an effective amount of exogenous MG53 sufficient to increase muscle satellite cell proliferation.

The inventors also determined that chronic systemic elevation of MG53 can increase the lifespan of a subject as compared to the expected lifespan of a subject of the same species and having substantially the same demographic profile with respect to gender and overall health. A group of mice with the same demographic profile were divided into two groups: Group 1 wild type mice, and Group 2 mice with sustained elevation of MG53 in the blood circulation. All of the Group 2 mice lived to at least 32 months, but all of the Group 1 mice died before 32 months.

The present inventors have established the efficacy of exogenous rhMG53 toward improvement of tissue performance. The data herein indicate MG53 can be administered exogenously and prophylactically to a subject to improve tissue performance.

Accordingly, the invention provides a method of improving tissue performance, the method comprising administering to a subject, one or more dosage forms that provide or induce expression of a prophylactically effective amount of MG53 in the subject, whereby the MG53 is taken up by said tissue.

It is by administration of exogenous MG53, by way of a dosage form comprising MG53 or causing expression of MG53 or releasing MG53, that tissue performance can be improved. It is also by administration of MG53, by way of a bioengineered dosage form comprising MG53 or expressing MG53, that tissue performance can be improved.

Suitable concentrations of MG53 in a dosage form include at least 1 ng of MG53/ml, at least 5 ng of MG53/ml, at least 10 ng of MG53/ml, at least 25 ng of MG53/ml, at least 50 ng of MG53/ml, at least 75 ng of MG53/ml, at least 100 ng of MG53/ml, at least 250 ng of MG53/ml, at least 500 ng of MG53/ml, at least 750 ng of MG53/ml, at least 1 μg of MG53/ml, at least 5 μg of MG53/ml, at least 10 μg of MG53/ml, at least 15 μg of MG53/ml, at least 20 μg of MG53/ml, at least 25 μg of MG53/ml, at least 30 μg of MG53/ml, at least 50 μg of MG53/ml, or at least 100 μg of MG53/ml. Higher concentrations are also acceptable, particularly in view the efficacy dose-response trend observed for MG53. These doses can be administered on a frequency as described herein or as determined to be most effective.

Suitable doses of MG53 that can be administered to a subject in one or more dosage forms include at least 1 ng of MG53, at least 5 ng of MG53, at least 10 ng of MG53, at least 25 ng of MG53, at least 50 ng of MG53, at least 75 ng of MG53, at least 100 ng of MG53, at least 250 ng of MG53, at least 500 ng of MG53, at least 750 ng of MG53, at least 1 μg of MG53, at least 5 μg of MG53, at least 10 μg of MG53, at least 15 μg of MG53, at least 20 μg of MG53, at least 25 μg of MG53, at least 30 μg of MG53, at least 50 μg of MG53, or at least 100 μg of MG53. Such doses can be on a total body weight basis or a per kg of body weight basis.

The invention also provides a method of improving tissue performance by systemically or locally administering to a subject a bioengineered cell (such as a MSC) and/or a bioengineered viral vector (such as a retroviral vector) to cause increased expression of MG53 in the blood (circulatory system) of said subject. Following administration to the subject, the bioengineered SC will express MG53 in said subject. Likewise, the viral vector will either express or induce expression of MG53 in said subject. The bioengineered MSC and/or viral vector may be administered to intramuscularly, intravenously, subcutaneously, orally, hepatically, or systemically.

The amount of therapeutic compound (MG53) incorporated in each dosage form will be at least one or more unit doses and can be selected according to known principles of pharmacy. An effective amount of therapeutic compound is specifically contemplated. By the term “effective amount”, it is understood that, with respect to, for example, pharmaceuticals, a pharmaceutically (therapeutically) effective amount is contemplated. A pharmaceutically effective amount is the amount or quantity of a drug or pharmaceutically active substance which is sufficient to elicit the required or desired therapeutic response, or in other words, the amount which is sufficient to elicit an appreciable biological response when administered to a patient.

The term “unit dosage form” is used herein to mean a dosage form containing a quantity of the drug, said quantity being such that one or more predetermined units may be provided as a single therapeutic administration.

The dosage form is independently selected at each occurrence from the group consisting of liquid solution, suspension, gel, cream, ointment, slab gel, insert (implant), syringe, or other known dosage form(s).

The dosage form can also include autologous blood serum. Dosage forms comprising autologous (blood) serum can be made as described by Geerling et al. (“Autologous serum eye drops for ocular surface disorders” in British Journal of Ophthalmology (2004) 88:1467-1474; dx.doi.org/10.1136/bjo.2004.044347 or by Fox et al. (Beneficial effect of tears made with autologous serum in patients with keratoconjunctivitis sicca in Arthritis Rheum. (1984), 28:4594611 the entire disclosures of which are hereby incorporated by reference, or as described herein (Example 16). In sonic embodiments, exogenous MG53 is added to the dosage forms, or stem cells expressing MG53 are added to the dosage forms, or viral vectors that cause cells to express MG53 are added to the dosage forms, or embodiments of two or more such systems are employed in said dosage form(s).

Accordingly, the invention provides an autologous serum dosage form comprising exogenously added MG53. The invention also provides an autologous serum dosage form comprising cells that express MG53. The invention also provides an autologous serum dosage form comprising a viral vector that causes cells to express MG53.

Compositions and dosage forms of the invention can further comprise one or more pharmaceutically acceptable excipients. Dosage forms can comprise one or more excipients independently selected at each occurrence from the group consisting of acidic agent, alkaline agent, buffer, tonicity modifier, osmotic agent, water soluble polymer, water-swellable polymer, thickening agent, complexing agent, chelating agent, penetration enhancer. Suitable excipients include U.S.F.D.A. inactive ingredients approved for use in parenteral or oral formulations (dosage forms), such as those listed in the U.S.F.D.A.'s “Inactive Ingredients Database (available on the following web site: www.fda.gov/Drugs/InformationOnDrugs/ucm113978.htm; October 2018), the entire disclosure of which is hereby incorporated by reference.

One or more antioxidants can be included in a composition of dosage form of the invention. Exemplary antioxidants include SS-31, NAC, glutathione, selenium, vitamin A, vitamin C, vitamin E, co-enzyme Q10, resveratrol, other GRAS antioxidant, or a combination of two or more thereof.

One or more zinc salts can be included in a composition or dosage form of the invention. Such zinc salt(s) may also be administered to a subject receiving exogenous MG53 or expressed MG53. Pharmaceutically acceptable zinc salts include Zinc gluconate, Zinc acetate, Zinc sulfate, Zinc picolinate, Zinc orotate, Zinc citrate, and other such salts comprising a zinc cation and organic or inorganic anion(s).

It should be understood, that compounds used in the art of pharmaceutical formulations generally serve a variety of functions or purposes. Thus, if a compound named herein is mentioned only once or is used to define more than one term herein, its purpose or function should not be construed as being limited solely to that named purpose(s) or function(s).

As used herein, “pharmaceutically acceptable salts” refer to derivatives of the disclosed compounds wherein the compound is modified by making an acid or base salt thereof. Examples of pharmaceutically acceptable salts include, but are not limited to, mineral or organic acid salts of basic residues such as amines; alkali or organic salts of acidic residues such as carboxylic acids; and others known to those of ordinary skill. The pharmaceutically acceptable salts can be synthesized from the parent therapeutic compound which contains a basic or acidic moiety by conventional chemical methods. Lists of suitable salts are found in Remington's Pharmaceutical Sciences, 17th ed., Mack Publishing Company, Easton, Pa., 1985, p. 1418, the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

MG53 can be used in cotherapy or adjunctive therapy with one or more other active ingredients to improve tissue function. Exemplary suitable active ingredients include, among others, U.S.F.D.A. approved drugs for parenteral or oral dosage forms.

The therapeutically acceptable dose, maximum tolerated dose (MTD), and minimally effective dose (MED) for each of said active ingredients is well known and set forth in the respective U.S.F.D.A. approved product package insert for each said active ingredients.

A composition, dosage form or formulation of the invention can include one, two or more active ingredients in combination with MG53. The dose of each said active ingredient in said composition, dosage form or formulation of the invention will be a therapeutically effective dose including and above the MED and including and below the MTD.

In some embodiments, the combination treatment of MG53 with another active ingredient provides at least additive therapeutic efficacy. In some embodiments, said combination provides synergistic therapeutic efficacy. In some embodiments, MG53 reduces the occurrence of, reduces the level of, or eliminates adverse events caused by the other active ingredient.

The acceptable concentrations of said excipients are well known in the art and specific concentrations (amounts) thereof are set forth in the package insert or package label of known commercial products containing the same.

It should be understood, that compounds used in the art of pharmaceutics may serve a variety of functions or purposes. Thus, if a compound named herein is mentioned only once or is used to define more than one term herein, its purpose or function should not be construed as being limited solely to that named purpose(s) or function(s).

In the examples below, ranges are specified for the amount of each ingredient. Ranges including “0” as the lowest value indicate an optional ingredient. The lower limit “>0” indicates the respective material is present.

As used herein, the terms “about” or “approximately” are taken to mean a variation or standard deviation of ±10%, ±5%, or ±1% of a specified value. For example, about 20 mg is taken to mean 20 mg±10%, which is equivalent to 18-22 mg.

As used herein, the term “prodrug” is taken to mean a compound that, after administration, is converted within a subject's body, e.g. by metabolism, hydrolysis, or biodegradation, into a pharmacologically active drug. The prodrug may be pharmacologically active or inactive. For example, a prodrug of MG53 (native or mutant) would be converted to the native form or mutant form, respectively, of MG53. The term “precursor” may also be used instead of the term “prodrug”.

As used herein, the term “derivative” is taken to mean: a) a chemical substance that is related structurally to a first chemical substance and theoretically derivable from it; b) a compound that is formed from a similar first compound or a compound that can be imagined to arise from another first compound, if one atom of the first compound is replaced with another atom or group of atoms; c) a compound derived or obtained from a parent compound and containing essential elements of the parent compound; or d) a chemical compound that may be produced from first compound of similar structure in one or more steps. For example, a derivative may include a deuterated form, oxidized form, dehydrated, unsaturated, polymer conjugated or glycosilated form thereof or may include an ester, amide, lactone, homolog, ether, thioether, cyano, amino, alkylamino, sulfhydryl, heterocyclic, heterocyclic ring-fused, polymerized, pegylated, benzylidenyl, triazolyl, piperazinyl or deuterated form thereof.

In the examples below, ranges are specified for the amount of each ingredient. Ranges including “0” as the lowest value indicate an optional ingredient. Compositions with quantities of ingredients falling within the compositional ranges specified herein were made. Compositions of the invention comprising quantities of ingredients falling within the compositional ranges specified herein operate as intended and as claimed.

In view of the above description and the examples below, one of ordinary skill in the art will be able to practice the invention as claimed without undue experimentation. The foregoing will be better understood with reference to the following examples that detail certain procedures for the preparation and use of compositions according to the present invention. All references made to these examples are for the purposes of illustration. The following examples should not be considered exhaustive, but merely illustrative of only a few of the many embodiments contemplated by the present invention. The methods described herein can be followed to prepare and use compositions of the invention and to practice methods of the invention.

### EXAMPLE 1

### rhMG53 Protein Production and Quality Control

The following process was used to produce recombinant human MG53 protein.

E. coli fermentation was used to obtain high quality (>97% purity) rhMG53 (recombinant human MG53) protein as described by Zhu et al. (“Polymerase transcriptase release factor (PTRF) anchors MG53 protein to cell injury site for initiation of membrane repair” in The Journal of biological chemistry (2011), 286, 12820-12824) and Weisleder et al. (Recombinant MG53 protein modulates therapeutic cell membrane repair in treatment of muscular dystrophy. Science translational medicine (2012), 4, 139ra185), the entire disclosures of which are hereby incorporated by reference. The membrane protective activity of rhMG53 from each preparation was determined with established micro-glass bead injury assay as described previously (ibid).

### EXAMPLE 2

### Determination of Intracellularly Aggregated MG53 by Immunohistochemical Staining

Immunofluorescent staining was performed as follows: slides were deparaffinized and rehydrated by incubating successively in xylene, 100% ethanol, 95%, 75%, 50% ethanol and PBS. Antigen retrieval was achieved by heating in the pressure cooker with Tris-EDTA buffer for 13 mins. Primary anti-MG53 antibody were applied and incubated at 4° C. overnight. Goat anti-rabbit/mouse secondary antibody Alexa-546/Alexa-647 were applied and incubated at room temperature for 1 h. All images were captured by Zeiss LSM 780 confocal microscope and analyzed by ImageJ.

### EXAMPLE 3

### Western Blot Analysis

For western blot, crude extracts from dissected muscle or heart of experimental animals were washed twice with ice-cold PBS and lysed in RIPA buffer (10 mM Tris-HCl, pH 7.2, 150 mM NaCl, 1% NP-40, 0.5% SDS, and 0.5% deoxycolate), supplemented with a cocktail of protease inhibitors (Sigma) and phosphatase inhibitors (Thermo Scientific). Heart, muscle lysates or serum samples were separated by 10% SDS-PAGE and transferred onto polyvinylidene fluoride membranes (PVDF) (Millipore). The blots were washed with Tri s-buffered saline Tween-20 (TB ST), blocked with 5% milk in TBST for 1 hour, and incubated with custom-made monoclonal anti-MG53 antibody and secondary antibody for 2 hours. Immunoblots were visualized with an ECL plus kit (Pierce).

### EXAMPLE 4

Following successful anesthesia induction, the sciatic nerve was exposed under a dissecting microscope and gently elevated on bipolar platinum hook electrodes to allow relatively isolated stimulation. The incision was extended to the dorsal surface of the leg, and the gastrocnemius muscle was exposed for single fiber electromyography. An uninsulated monopolar ground electrode was placed in the opposite flank. Low-amperage (1-10 mA) square-wave pulses of 50-μs duration were delivered at 2 HZ to the stimulating electrodes with a variable intensity stimulator (Oxford/Teca Corp., Pleasantville, N.Y.). A standard 25-mm single fiber needle electrode (Oxford/Teca) was placed longitudinally in the gastrocnemius muscle and carefully positioned to record single fiber discharges. Signals were recorded on a computerized EMG system (Neuroscan Medical Systems, Sterling, Va.) utilizing proprietary software at filter settings of 500 HZ to 10 kHZ, a sweep of 0.5 ms/division, and sensitivities of 0.2-2 mV/division.

### EXAMPLE 5

Since C57BL/6J mice displayed a sharp rise in jitters starting at a transition from 24 to 27 months age, we therefore treated mice at 24 months with rhMG53. The mice are administered rhMG53 (6mg/kg, subcutaneous) over the 6-week period of treatment.

### EXAMPLE 6

Mice underwent triceps surae plantarflexion torque assessment with an in vivo muscle contractility apparatus (Model 1300A; Aurora Scientific, Aurora, Ontario, Canada; Supp. Info. FIG. 1, Supp. Info. Methods) as previously detailed. 36 Briefly, the right hind paw was taped to the force sensor and positioned at 90°. The hind limb was extended to position the knee in the locking position and secured at the femoral condyles. Two disposable monopolar electrodes (Natus Neurology, Middleton, Wis.) were inserted near the tibial nerve, just posterior to the knee. Maximum plantarflexion twitch torque was recorded after a single, supramaximal stimulation (200-μs square wave pulse). Maximum tetanic contraction torque was assessed after a train of supramaximal square wave stimulations of 200-μs duration delivered at 125-HZ stimulation frequency.

### EXAMPLE 7

The soleus muscle was collected from mice for endpoint studies and fixed in 4% paraformaldehyde at room temperature (RT) for 30 min. 42, 43 Muscles were teased into fibers by using size 55 forceps (Fine Science Tools, North Vancouver, British Columbia, Canada) and then incubated in blocking buffer (10% goat serum/4% bovine serum albumin/3% Triton-X 100/phosphate-buffered saline (PBS)) at RT for 2 h. An overnight primary antibody (α-NF-200, 1:5,000, Ab72996; Abcam, Cambridge, Mass.) incubation at 4° C. was performed, followed by three 10-min washes with PBS before receiving a 2-h incubation with secondary antibody (Alexa Fluor 594 goat α-chicken, 1:1,000, A11042; Life Technologies, Grand Island, N.Y.) and α-α-bungarotoxin-488 (1:1,000, B13422; Life Technologies) at RT. Samples then underwent three 10-min washes with PBS at RT before being mounted onto Superfrost positively charged glass slides (Fisher Scientific, Pittsburg, Pa.) and sealed with Fluoromount-G (Southern Biotech, Birmingham, Ala.). Samples were imaged at ×20 and ×40 with a confocal microscope (DM IRE2; Leica, Wetzlar, Germany) with Leica software (version 2.1). Images were viewed in FIJI (LOCI; University of Wisconsin-Madison, Madison, Wis.).

### EXAMPLE 8

Male C57BL/6N mice at 12-14 weeks of age were individually housed in a 12:12 hour (dark: light) cycle, and acclimated to the vivarium for 1 week prior to initiation of muscle injury and intervention. Animals were randomized to treatment groups based on baseline body weight. Mice were anesthetized using isoflurane. Hindlimb muscles were stimulated to produce tetanic contractions (via sciatic nerve; 60 repetitions; 10 seconds apart) while being forcibly lengthened in vivo. The protocol was approved by the IACUC. Following eccentric contraction-induced muscle injury, mice were divided into groups of 10 each according to the following experimental designs: tail vein administration at 4 hours post muscle injury with different doses of rhMG53 (0, 0.6, 2, 6 and 20 mg/kg); and subcutaneous administration of rhMG53 (6 mg/kg) on a daily basis, with the first dose applied at 24 hours post muscle injury. Following the longitudinal study with repetitive dosing of rhMG53, mice were sacrificed at 28 days post injury, and serum glucose and triglycerides were quantified. All measurements were conducted in a double-blinded manner.

### EXAMPLE 9

Mouse echocardiographic images were obtained with a Vevo 2100 high frequency, high resolution (30 micron) digital imaging ultrasound system (VisualSonics, Inc.), which is equipped with 24 and 38 MHz Microscan transducers and linear array technology for B-mode and M-mode imaging and color Doppler mode scanning as previously described. Serial echocardiograms were obtained at baseline and every week till end of the study and were performed under isoflurane anesthesia (3% for induction and 1% for maintenance). Using a rectal temperature probe, body temperature was carefully maintained between 36.7 and 37.3° C. throughout the study. Digital images were analyzed off-line by blinded observers using the Vevo 2100 workstation software. At least three measurements were taken and averaged for each parameter. Standard echocardiographic parameters were derived from the two-dimensional, M-mode, and Doppler images.

### EXAMPLE 10

Blood samples were obtained by cardiac puncture technique at the time when mice were euthanized. Serum creatinine levels were measured by a rodent blood analyzer.

### EXAMPLE 11

### Treadmill and Voluntary Wheel-Running Test

tPA-MG53 and wild type littermates were initially trained (5 m/min running for 5 mins each time, running for 3 times each day for three days) on a small animal treadmill (Columbus Instruments). Then the mice were subjected to treadmill running at 10 m/min for 6 hours. Twenty hours after the initial exercise training, mice were subjected to running at 6, 8, 10, 12, 14, and 16 m/min each for 3 minutes on the treadmill to test the capacity of recovery from muscle injury. The number of times the mice fail to run forward and touch the bottom of the electric grid of the treadmill and remain there for over 7 seconds was recorded as drop-out. Drop-outs of each mouse at each different speed were recorded. In separate studies, tPA-MG53 and wild type littermates were individually kept in cages equipped with voluntary free-spinning running wheels (Columbus Instruments, Columbus, Ohio) for one week. The voluntary running activity were recorded by wheel rotations at 2-hour intervals using Windows software (Columbus Instruments, Columbus, Ohio).

### EXAMPLE 12

Flexor digitorum brevis (FDB) muscle fibers were isolated from wild type and tPA-MG53 mice following the protocol of Zhu et al 39. They were loaded with 10 μM Fura-2 AM. The ratio of Fura-2 fluorescence at excitation wavelength of 340 and 380 nm was measured using a PTI spectrofluorometer (Photon Technology International) to assess the changes in intracellular concentration [Ca²⁺]ᵢ following stimulation with KCl. Zero Ca²⁺ or 2 mM Ca²⁺ Tyrode's solution was perfused onto the fiber before adding 110 mM KC1 to induce Ca²⁺ store release.

### EXAMPLE 13

Extensor digitorum longus (EDL) muscle from mg53-/-, tPA-MG53 and their wild type littermates were dissected and digested with 0.2% collagenase at 35° C. for 45 min in a shaking water bath. Single muscle EDL muscle fibers were picked with a heat polished Pasteur pipette and placed at the center of the individual wells of a 24-well matrigel coated plate. The culture media (DMEM plus 20% FBS) was changed every 3 days to allow outgrowth of muscle satellite cells at 37° C. (5% CO₂). The identity of the cultured satellite cells were confirmed by Pax 7 antibody (Iowa Hybridoma Bank) staining by flow cytometry and immunofluorescent staining. Antibodies against MyoD (myoblast marker) and PDGFα (fibroblast marker) were used to show the purity of isolated satellite cells.

All values disclosed herein may have standard technical measure error (standard deviation) of ±10%. The term “about” or “approximately” is intended to mean ±10%, ±5%, ±2.5% or ±1% relative to a specified value, i.e. “about” 20% means 20±2%, 20±1%, 20±0.5% or 20±0.25%. The term “majority” or “major portion” is intended to mean more than half, when used in the context of two portions, or more than one-third, when used in the context of three portions. The term “minority” or “minor portion” is intended to mean less than half, when used in the context of two portions, or less than one-third, when used in the context of three portions. It should be noted that, unless otherwise specified, values herein concerning pharmacokinetic or dissolution parameters are typically representative of the mean or median values obtained.

The above is a detailed description of particular embodiments of the invention. It will be appreciated that, although specific embodiments of the invention have been described herein for purposes of illustration, various modifications may be made without departing from the spirit and scope of the invention. Accordingly, the invention is not limited except as by the appended claims. All of the embodiments disclosed and claimed herein can be made and executed without undue experimentation in light of the present disclosure.

1) A method of improving tissue function, said method comprising administering to a subject exogenous MG53 in an amount effective to improve said tissue function as compared to the performance of said tissue in the absence of said exogenous MG53, wherein said tissue is not diseased and has not been physically injured by impact force, puncture, burning, irradiation, or cutting.

2) The method of claim 1, wherein administration of exogenous MG53 improves contraction of muscle tissue, improves Ca²⁺ signaling in muscle tissue, improves muscle satellite cell proliferation, improves recovery of strenuously exercised muscle, improves overall performance of muscle tissue, improves heart output function, improves neuromuscular junction integrity, improves brain function, provides a reduction in kidney resident immune cell activation, provides a reduction in macrophage infiltration, and/or provides increased kidney tubular health.

3) The method of claim 1, wherein said tissue a) is healthy except for exhibiting reduced (impaired) function as compared to other similar tissue; or b) is healthy and administration of MG53 improves function (performance) of said tissue as compared to function (performance) prior to administration of MG53 to said tissue.

4) The method of claim 1, wherein said tissue a) exhibits increased intracellular aggregation of MG53 prior to said administering; or b) exhibits elevated levels of intracellular MG53 aggregation prior to said administering.

5) The method of claim 1, wherein said exogenous MG53 is administered chronically over a period of at least 1-6 weeks.

6) The method of claim 1, further comprising the step of administering to said subject at least one antioxidant and/or at least one zinc salt.

7) The method of claim 6, wherein a) the molar ratio of MG53 to antioxidant ranges from 0.01 to 10; and/or b) the molar ratio of zinc salt to MG53 is at least about 2:1.

8) A method of improving athletic performance in a human or animal subject, the method comprising at least the following step(s): prior to conducting an athletic activity, administering to said subject an effective amount of MG53, whereby said administering results in improved athletic performance of said subject as compared to said subject's performance in said athletic activity when not administered MG53.

9) The method of claim 8 further comprising the step of said subject conducting said athletic activity, and MG53 is administered acutely or chronically before said athletic activity.

10) The method of claim 9 further comprising the step of administering one or more other active ingredients to said subject to further improve athletic performance.

11) The method of claim 9, further comprising the step of administering to said subject at least one antioxidant and/or at least one zinc salt.

12) The method of claim 11, wherein a) the molar ratio of MG53 to antioxidant ranges from 0.01 to 10; and/or b) the molar ratio of zinc salt to MG53 is at least about 2:1.

13) The method of claim 8, wherein administration of exogenous MG53 improves contraction of muscle tissue, improves Ca²⁺ signaling in muscle tissue, improves muscle satellite cell proliferation, improves recovery of strenuously exercised muscle, improves overall performance of muscle tissue, improves heart output function, improves neuromuscular junction integrity, improves brain function, provides a reduction in kidney resident immune cell activation, provides a reduction in macrophage infiltration, and/or provides increased kidney tubular health.

14) A method of improving recovery of strenuously exercised muscles in a human or animal subject, the method comprising at least the following step(s): prior to or after conducting a strenuous athletic activity, administering to said subject an effective amount of MG53, whereby said administering results in improved recovery of said strenuously exercised muscle as compared to recovery when said subject is not administered MG53.

15) The method of claim 14 further comprising the step of said subject conducting strenuous exercise, and MG53 is administered acutely or chronically before or after said strenuous athletic activity.

16) The method of claim 15, further comprising the step of administering one or more other active ingredients to said subject to further improve said recovery of strenuously exercised muscle.

17) The method of claim 14, further comprising the step of administering to said subject at least one antioxidant and/or at least one zinc salt.

18) The method of claim 17, wherein a) the molar ratio of MG53 to antioxidant ranges from 0.01 to 10; and/or b) the molar ratio of zinc salt to MG53 is at least about 2:1.

19) The method of claim 14, wherein administration of exogenous MG53 improves contraction of muscle tissue, improves Ca²⁺ signaling in muscle tissue, improves muscle satellite cell proliferation, improves recovery of strenuously exercised muscle, improves overall performance of muscle tissue, improves heart output function, improves neuromuscular junction integrity, provides a reduction in macrophage infiltration, and/or provides increased kidney tubular health.
